SEQUENCE LISTING (10 sequences)

SEQ ID NO: 1 - DNA, 21 bp, Artificial
  primer for human insulin
gcctttgtgaaccaacacctg

SEQ ID NO: 2 - DNA, 21 bp, Artificial
  primer for human insulin
gttgcagtagttctccagctg

SEQ ID NO: 3 - DNA, 19 bp, Artificial
  primer for IPF1
cccatggatgaagtctacc

SEQ ID NO: 4 - DNA, 19 bp, Artificial
  primer for IPF1
gtcctcctcctttttccac

SEQ ID NO: 5 - DNA, 26 bp, Artificial
  primer for Ngn3
ctcgagggtagaaaggatgacgcctc

SEQ ID NO: 6 - DNA, 27 bp, Artificial
  primer for Ngn3
acgcgtgaatgggattatggggtggtg

SEQ ID NO: 7 - DNA, 21 bp, Artificial
  primer for b-actin
catcgtgggccgctctaggca

SEQ ID NO: 8 - DNA, 23 bp, Artificial
  primer for b-actin
ccggccagccaagtccaggacgg

SEQ ID NO: 9 - DNA, 29 bp, Artificial
  primer for exon 1 of human Insulin
gcggagctctctcctggtctaatgtggaa

SEQ ID NO: 10 - DNA, 29 bp, Artificial
  primer for exon 1 of human Insulin
gcgctcgagctcttctgatgcagcctgtc

(19) United States
(12) Patent Application Publication
(10) Pub. No.: US 2004/0191901 A1
(43) Pub. Date: Sep. 30, 2004

(54) Insulin producing cells derived from human embryonic stem cells

(76) Inventors: Suheir Assady, Nazareth (IL); Gila Maor, Kiriat Motzkin (IL); Michal Amit, Misgav (IL); Joseph Itskovitz-Eldor, Haifa (IL); Karl L. Skorecki, Kiryat Shmuel (IL); Maty Tzukerman, Hagetaot (IL)

Correspondence Address: WINSTON & STRAWN, PATENT DEPARTMENT, 1400 L STREET, N.W., WASHINGTON, DC, 20005-3502, US

(21) Appl. No.: 10/714,348

(22) Filed: Nov. 14, 2003

Related U.S. Application Data
(63) Continuation of application No. PCT/IL02/00369, filed on May 14, 2002.
(60) Provisional application No. 60/328,798, filed on Oct. 15, 2001.

(30) Foreign Application Priority Data
May 15, 2001 (IL) 143155

(51) Int. Cl. C12N 5/08

(52) U.S. Cl. 435/366

(57) ABSTRACT

The present invention discloses insulin producing cells derived from human embryonic stem cells. Methods of generating, enriching, selecting, cloning and isolating populations of insulin-producing embryonic cell lines are disclosed. Insulin production in these cells can be glucose responsive or otherwise regulatable. Advantageously, these insulin producing cells can be stable cell lines derived from stable transfectants of hESC. Methods of using these cells, cell populations or cell lines particularly for cell replacement therapy are also disclosed.

### FIELD OF THE INVENTION

The present invention relates to insulin-producing human embryonic stem cells, to the generation and enrichment of populations of insulin-producing human embryonic stem cells, to isolation of insulin-producing human embryonic stem cells or stable cell lines and to methods of using these cells, particularly for cell replacement therapy.

### BACKGROUND OF THE INVENTION

Type 1 diabetes mellitus generally results from autoimmune destruction of pancreatic islet β-cells, with consequent absolute insulin deficiency and complete dependence upon exogenous insulin treatment. The relative paucity of donations for pancreas or islet allograft transplantation has prompted the search for alternative sources for β-cell replacement therapy.

Recent studies have emphasized the importance of strict glycemic control in order to reduce ophthalmologic, neurologic, and renal complications of Type I diabetes mellitus (Fioretto et al., 1998). Yet, pancreatic and islet cell replacement is currently considered to be the only truly curative approach. Indeed, this approach was recently shown to reverse glomerular lesions in patients with diabetic nephropathy (Shapiro et al., 2000). The promise of this approach has recently been further strengthened by a report of the use of an improved, less hazardous glucocorticoid-free immunosuppresive regimen in islet allograft transplantation (Samstein et al, 2001). However, the shortage in donations is a primary obstacle, preventing this approach from becoming a practical solution. Thus, attention has focused on the use or alternative sources such as xenografts, which have other disadvantages, including the potential risk of undetermined zoonotic infections (Efrat, 1998). It has also been suggested that β-cell lines derived from rodents might provide an unlimited source for cell replacement therapy. In addition to the problem inherent in xenobiotic sources, such cell lines have been shown to display phenotypic instability, with loss of insulin biosynthesis and regulated secretion while proliferating (Efrat, 1999; Soria et al., 2000a; Cheung et al., 2000). Another more recently described approach involves extending the β-cell phenotype to other tissues using in vivo gene transfer (Lee et al., 2000; Ferber et al., 2000), either by expressing the insulin gene or an insulin gene analogue under the control of a glucose sensitive promoter, or by ectopic expression of insulin promoter factor1/pancreatic and duodenal homeobox gene 1 (IPF1/PDX1) (Thomson et al., 1998).

The establishment of pluripotent human embryonic stem (hES) cells (Reubinoff et al., 2000; Shamblott et al., 1998) and embryonic germ (EG) cells (Soria et al., 2000b), have introduced a new potential source for cell therapy in type 1 diabetic patients, especially in the light of recent successes in producing glucose-sensitive insulin-secreting cells from mouse ES (Shamblott et al., 2001). hES cells grow as homogeneous and undifferentiated colonies, when they are propagated on a feeder layer of mouse embryonic fibroblasts (MEFs; Reubinoff et al., 2000). As previously shown, they have a normal karyotype, express telomerase and embryonic cell surface markers. Removal from the MEF feeder layer is associated with differentiation into derivatives of the three embryonic germ layers, as evident from teratomas formed following subcutaneous injection in nude mice (Reubinoff et al., 2000). Endodermal markers, but not insulin expression, were reported in a previous general survey of different growth conditions and differentiation markers in EG cells (Schuldiner et al., 2000). Using RT-PCR applied to RNA extracted from differentiated hES cells, detection of a variety of differentiated cell markers including insulin was reported (Robertson, 1987).

A method of enriching a mixed population of mammalian cells for mammalian stem cells is disclosed in U.S. Pat. No. 6,146,888. According to the method the mixed population of mammalian cells comprises an antibiotic resistance gene operatively linked to a promoter which preferentially expresses the antibiotic gene in mammalian stem cells. Accordingly, the mixed population of mammalian cells is cultured in vitro under conditions conducive to preferential survival of mammalian stem cells in the presence of antibiotics.

Various methods for generating novel embryonic cell populations, for propagation of embryonic stem cells utilizing combinations of growth factors and for immortalization of such cell lines are known in the art, as for example disclosed in U.S. Pat. Nos. 5,690,926, 5,753,506 and 6,110,739 and in the European Patent Application No. 380646, among many others.

A method for culturing human embryonic stem cells in vitro for prolonged maintenance while preserving the pluripotent character of these cells, as well as a purified preparation of said cells, is disclosed in U.S. Pat. No. 6,200,806. The embryonic stem cells also retain the ability, throughout the culture and after continuous culture for eleven months, to differentiate into all tissues derived from all three embryonic germ layer.

A method for producing a surface for supporting the growth, attachment and/or differentiation of cells, including stem cells, is disclosed in U.S. Pat. No. 6,232,121. The method comprises growing osteosarcoma cells on a surface under conditions that promote the secretion by the cells of an extracellular matrix comprising at least one biologically active growth factor, while the extracellular matrix and the growth factor are concomitantly produced and the extracellular matrix is attached to said surface.

A method for regenerating in mammals a desired living tissue of a desired configuration from undifferentiated embryonic stem cells, is disclosed in U.S. Pat. No. 6,328,765.

A method for treating a human subject by administering a therapeutically effective amount of human mesenchymal stem cells, is disclosed in U.S. Pat. No. 6,355,239. The stem cells according to this patent, may express incorporated genetic material of interest.

Nowhere in the background art is it taught that insulin-producing cell lines may be derived from established human stem cell lines.

### SUMMARY OF THE INVENTION

It is an object of the present invention to provide insulin-producing cells derived from human embryonic stem cells.

It is yet another object of the present invention to provide cell populations enriched for insulin-producing cells derived from human embryonic stem cells. Preferably cell populations containing selected insulin-producing cells derived from human embryonic stem cells, more preferably isolated cells or most preferably cloned cell lines will be provided in accordance with the principles of the present invention.

According to another aspect of the present invention insulin-producing cells derived from hES cells or cell populations comprising insulih-producing cells deived from hES cells will be regulatable. Preferably they will be glucose responsive, or glucose regulatable in terms of the insulin production and secretion.

According to yet another aspect of the present invention the insulin-producing cells derived from hES cells or cell populations comprising insulin-producing cells derived from hES cells will be stable over prolonged periods of time, preferably they will be long-lived, i.e., will not undergo growth arrest or senescence, more preferably they will be stable cell lines, most preferably they will be stable clonal cell lines, alternatively they will be immortalized cell lines.

According to yet another aspect according to the present invention, the insulin producing cells derived from hES cells or cell populations comprising insulin-producing cells derived from hES cells will be useful for medical applications, including but not limited to cell replacement therapy.

According to the principles of the invention it is now disclosed that pluripotent undifferentiated human embryonic stem (hES) cells can serve as a system for lineage specific differentiation. In a currently preferred embodiment using hES cells in both adherent and suspension culture conditions, it was demonstrated that in vitro differentiation included the generation of cells with characteristics of insulin producing β-cells. Immunohistochemical staining for insulin was observed in a surprisingly high percentage of cells. Secretion of insulin into the medium, was observed in a differentiation-dependent manner, and was associated with the appearance of other β-cells markers. These findings validate the hES cell system, as a potential basis for derivation and/or enrichment of human β-cells or their precursors as a possible source for cell replacement therapy in diabetes.

According to a currently more preferred embodiment of the invention it is now disclosed that it is possible to stably transfect human embryonic stem cells with the insulin promoter. As will be exemplified hereinbelow, these stable transfectants maintain their pluripotency.

According to one currently most preferred embodiment the insulin promoter may be linked to a convenient marker such as a fluorescent marker, such as green fluorescent protein by way of non-limiting example, which enables the transfectants to be selected or enriched by fluorescent activated cell sorter (FACS). The stable transfectants can be selected with antibiotics, such as hygromycin by way of non-limiting example, and be successfully cloned.

### BRIEF DESCRIPTION OF THE DRAWINGS

FIG. 1A demonstrates an embryonic body (EB), 20, in suspension 3 days after removal of ES cells from MEF.

FIG. 1B shows an EB, x40, in suspension 3 days after removal of ES cells from MEF.

FIG. 1C represents an EB, x40, in suspension 17 days after removal of ES cells from MEF.

FIG. 2A displays insulin expression in control tissue of normal human pancreas.

FIG. 2B-D exhibits insulin expression in an EB at day 19 after differentiation.

FIG. 2E shows the cytoplasmic localization of staining (insulin expression) in an EB, 19 days after differentiation.

FIG. 2F demonstrates EBs at day 19 after differentiation with non-immune serum.

FIG. 3A presents insulin secretion by undifferentiated hES cells (uhES) cultured in knockout medium, or allowed to differentiate in high-density adherent conditions (dhES) for 22 and 31 days.

FIG. 3B exhibits insulin secretion by hES grown in culture and in suspension as embryoid bodies for 20-22 days at varying glucose concentrations.

FIG. 4A demonstrates expression of β-cell related genes: insulin and GK in the total RNA of undifferentiated hES (uhES), differentiated hES growing either as EB or as high-density adherent cell cultures (dhES), and from normal human fibroblasts (NHF)

FIG. 4B exhibits expression of Glut-2 and Glut-1 in the total RNA of undifferentiated hES (uhES), EBs, high-density adherent cell cultures (dhES), and normal human fibroblasts (NHF).

FIG. 4C shows expression of Oct4, Ngn3 and IPF1/PDX1 in the total RNA of undifferentiated hES (uhES), EBs, high-density adherent cell cultures (dhES), and normal human fibroblasts (NHF).

FIG. 5A-B presents EBs generated from clones stably transfected with an insulin promoter.

FIG. 5C-D presents insulin positive cells within EBs generated from clones stably transfected with an insulin promoter.

FIG. 6A shows EGFP fluorescence after differentiation in IIB6 clone.

FIG. 6B exhibits EGFP fluorescence after differentiation in IB3 clone.

FIGS. 7A and 7B demonstrate EGFP expressing cells surrounded by neural projections.

### DETAILED DESCRIPTION OF THE PREFERRED EMBODIMENTS

Recently, islet cells were successfully generated in vitro from pancreatic stem cells (Ramiya et al., 2000; Amit et al., 2000) of humans and adult mice. In the latter study, Ramiya et al. have shown that these islet cells could reverse diabetes after being implanted in non-obese diabetic mice. However, the major practical limitation of this approach is the restricted number of cells that can be cultivated from human pancreata. Hence, human embryonic stem cells represent a reasonable potential alternative.

The present invention relates to the generation, enrichment, selection, cloning and usage of insulin-producing cells derived from human embryonic stem cells. The invention will now be described in detail with respect to preferred embodiments.

### I. Definitions

A “pluripotent embryonic stem cell” as used herein means a cell which can give rise to many differentiated cell types in an embryo or adult, including the germ cells (sperm and eggs). It is the least differentiated cell in a cell lineage. Pluripotent embryonic stem cells are also capable of self-renewal. This cell type is also referred to as an “ES cell” herein. However, stem cell is an operational term. It is known that ES cells will only retain the stem cell phenotype in vitro when cultured on a feeder layer or when cultured in medium conditioned by certain cells. In the absence of feeder layer or conditioned medium, the ES cells spontaneously differentiate into a wide variety of cell types, resembling those found during embryogenesis and in the adult animal.

“Feeder layers” as used herein can either be cells or cell lines cultured for the purpose of culturing pluripotent ES cells. Alternatively, feeder layers can be derived from or provided by the organ or tissue in which the primordial germ cells, embryonic ectoderm cells or germ cells are located, e.g. the gonad. Thus, if the somatic cells of the tissue or organ in which the desired cells are located are sufficient to provide the appropriate culture environment, a separate feeder layer is not required. Alternatively, the feeder cells could be substituted with extracellular matrix plus bound growth factors.

The term “supplements” is interchangeably used herein with the term “factors”. Factors added to the culture medium are essential to the formation of pluripotent ES cells. Thus, the amount of the factors utilized is determined by the end result of the pluripotent ES cells. However, the factors also serve to enhance the growth and allow the continued proliferation of the cells. Accordingly, the factors also appear to help the cells survive.

The term “genetically modified cells” as referred to herein relates to cells being transfected by a vector, as exemplified by an expression vector comprising the coding sequence of a gene of interest, said cells capable of expressing said gene. Particularly in the context of this invention, the genetically modified cells are embryonic stem cells transfected with expression vector that contains at least one gene coding sequence selected from: internal ribosome entry site (IRES); a cell specific gene promoter, e.g. PDX-1 that becomes active in β-cells progenitors at a very early stages of β-cells differentiation pathway; human telomerase reverse transcriptase (hTERT); a selection marker including but not limited to antibiotic, e.g. neomycin, or fluorescence marker, e.g. enhanced green fluorescent protein (EGFP); a constitutive promoter, e.g. PGK promoter or other mammalian promoter. The genetically modified hES cells may be cultured under conditions that will direct them to differentiate into insulin producing cells.

### II. Preferred Modes for Carrying Out the Invention

It is known in the art that embryonic development of the pancreas is the result of several distinct but interacting mechanisms involving growth factors, epithelial-mesenchymal interactions (Edlund, 1999) and extracellular matrix that eventually regulate the expression of diverse transcription factors (Apelqvist et al., 1999; Kim et al., 2001; Efrat, 1997). However, the initial signal in the cascade of events that eventuate in the commitment of gut endoderm to develop into pancreatic tissue is still unknown.

Currently used hES cells are not of clonal origin, despite their homogenous appearance in the undifferentiated state, suggesting the need to examine the in vitro differentiation of each hES derived cell line independently, or to examine clonal hES cell lines with well defined differentiated responses to growth factors (Klug et al., 1996).

The embryonic stem cells display the innate property to differentiate spontaneously. In order to enrich the population of the undifferentiated hES cells of the invention and to maintain its homogeneity, the innate spontaneous differentiation of these cells has to be suppressed. Methods for suppressing differentiation of embryonic cells may include culturing the undifferentiated embryonic cells on a feeder layer, such as of murine fibroblasts as in the non-limiting examples described hereinafter, or in media conditioned by certain cells.

Induction of differentiation in hES cells, preferably a controlled induction towards a specific cell lineage, is primarily achieved for example by removing the differentiation-suppressing element, e.g. the feeder layer, from the culture. To effectively control the consequent differentiation, the cells must be in a homogeneous state. Any cell culture media that can support the growth and differentiation of embryonic stem cells, can be used with the present invention. Such cell culture media include, but are not limited to Basal Media Eagle, Dulbecco's Modified Eagle Medium, Iscove's Modified Dulbecco's Medium, McCoy's Medium, Minimum Essential Medium, F-10 Nutrient Mixtures, OPTI-MEM® Reduced-Serum Medium, RPMI Medium, and Macrophage-SFM Medium or combinations thereof. The culture medium can be supplied in either a concentrated (e.g.: 10x) or non-concentrated form, and may be supplied as either a liquid, a powder, or a lyophilizate. Culture media is commercially available from many sources, such as GIBCO BRL (Gaithersburg, Md.) and Sigma (St Louis, Mo.)

According to one aspect of the present invention, controlled differentiation in vitro of hES cells towards insulin-producing cells, is preferably conducted under serum-free conditions, also termed hereinafter knockout medium. Preferably, the knockout medium is supplemented with serum replacements; nonessential amino acids; 2-mercaptoethanol; glutamine. Most preferably, the knockout medium is further supplemented with human recombinant basic fibroblast growth factor (hrbFGF).

ES cells can be used to screen for factors which produce ES derivative (more differentiated) cells. Many standard means to determine the presence of a more differentiated cell are well known in the art For example, RT-PCR applied to RNA extracted from differentiated hES cells, enabled detection of a variety of differentiated cell markers including insulin (Robertson, 1987). However, quantitative aspects, including elaboration of insulin into the medium, and percentage of insulin producing cells, were not known in the art Such information is crucial to enable the use of hES cells as a source for β-cell replacement cell therapy.

The present invention provides evidence that a pathway for producing insulin-secreting cells with additional β-cell features is not an infrequent outcome in the course of spontaneous differentiation of human embryonic stem cells in culture, under the appropriate conditions as disclosed herein. This observation is a prerequisite for experimental strategies based upon the enrichment of spontaneously appearing β-cells or their precursors for cell replacement therapy. The cells described herein, produce and secrete insulin, and express two essential genes, Glut-2 and islet-specific glucokinase (GK), that are believed to play an important role in β-cell function and glucose stimulated insulin secretion (Matschinsky et al., 1998; Shepherd et al., 1999; Alarcón et al., 1998). The possibility that the insulin staining cells are unrelated to β-cells and are of extraembryonic or other origin (Ling et al. 1996) is highly unlikely, in view of the other markers identified, including the temporal course of appearance of β-cell developmental markers and in view of the secretion of fully processed insulin.

Another prerequisite for effective cell replacement therapy that the insulin production by human embryonic stem cells be glucose responsive or otherwise regulatable. As long as β-cells are not in a homogeneous or enriched state, but rather present among other cell types, it is not possible to isolate the effects of glucose from countervening effects of other secretagogues. Furthermore, in the absence of homogeneous cell populations, comparisons based upon different experimental conditions are not readily quantified because of heterogeneity among EBs and the difficulty in normalizing insulin response to parameters such as protein or DNA content (Jonkers et al., 1999).

Preferred strategies for selecting progenitor cells, for the purpose of isolation and propagation of selected cells, from a heterogeneous ES cell population, include transfection of pluripotent ES cells with a cell specific promoter. Preferably, the cell specific promoter may be further linked to a convenient marker such as antibiotic which enables to select the antibiotic resistant clones or fluorescent marker which enables to select the desired cells by fluorescent activated cells sorter (FACS).

According to one currently preferred embodiment of the present invention non-differentiated human ES cells are transfected with a cell specific gene promoter that becomes active in β-cells progenitors at the very early stages of β-cells development wherein said transfection does not impair the pluripotent character of the ES cells. Additionally, a selection marker is added under the control of said promoter and transfected cells are allowed to propagate into differentiation resulting in the production of selective insulin-producing cell clones.

The fine-tuning of insulin secretion in response to glucose requires cross talk between adjacent β-cells due to functional heterogeneity, and it has been shove that isolated β-cells function differently compared to β-cells found in clusters or pseudoislets (Charollais et al., 2000; Bonner-Weir et al., 2000). Conceivably, long term exposure to high glucose levels might affect the function of such insulin-producing cells and reduce their responsiveness to acute glucose changes as has been previously reported in other systems (Jonkers et al., 1999). This high glucose medium is needed to maintain the viable growth of hES in culture, but this does not preclude the possibility that protocols allowing growth of cells with insulin-producing capability in media containing lower glucose concentrations, may impart or restore glucose responsiveness. In any case, for treatment of diabetes with β-cell grafts derived from differentiated hES, it is necessary to demonstrate stimulus-secretion coupling after obtaining enriched or homogeneous β-cell cultures, as has been demonstrated for mouse ES derived β-cells (Shamblott et al., 2001).

For the first time it is now disclosed that using the differentiation of the H9 line of hES cells under a variety of experimental approaches disclosed herein, abundant cells with β-cell features, including most notably insulin production and secretion are established. It is now disclosed for the first time that stable transfectants of hES have been obtained. These transfectants maintain pluripotency, may be selected, enriched and cloned. When these stably transfected clones were grown in suspension cultures, cell aggregates (embryoid bodies) were generated, similar in appearance to wild type hESC.

It is conceivable that enrichment and propagation of large number of undifferentiated as well as differentiated embryonic stem cells will suffer the disadvantage of low proliferation capacity and limited life span. Conditions and methods designed to overcome this limitation will greatly increase the clinical benefits of cell replacement therapy. This is true both for increased “self-renewal” of the pluripotent undifferentiated embryonic stem cells, which will provide a larger supply of hES cells, and for proliferation with differentiation, which will provide a larger supply of lineage-specific cells, particularly insulin-producing β cells. Agents which may confer growth advantage to differentiated- and undifferentiated embryonic stem cells will be preferably utilized in the present invention. Telomerase in particular may be used to maintain telomere length and integrity and thereby extend the proliferation capacity of the embryonic stem cells. In those instances where ectopic over-expression of human telomerase reverse transcriptase (hTERT) does not adversely influence the differentiation pattern, it is possible to generate a fully differentiated desired lineage of telomerase positive ES cells.

In terms of strategies for enriching the population of insulin-secreting hES derived cell lines for further characterization and study, our results certainly indicate that the approach first described by Klug et al. to enrich cardiomyocytes from mouse ES cells is potentially adapted to the present application (Klug et al., 1996). Briefly, Klug's approach is based upon a stable transfection of ES cells with a cardiac myosin promoter following induced differentiation of said cells towards cardiomyocytes. This simple genetic modification permitted the generation of essentially pure culture of cardiomyocytes from differentiating ES cells. As one non-limitative example, in the case of β-cells, the insulin promoter fused to a downstream selection marker could serve as the relevant selection tool for these cells from differentiated human ES cells. Indeed, recently this strategy was extended to enrichment of β cells from mouse ES cells (Shamblott et al., 2001). Our findings indicate that even under conditions of spontaneous differentiation from non-clonal pluripotent hES cells, EBs are produced with a surprisingly high representation of cells with insulin producing capacity. In the current study more than 60% of EBs contained scattered pockets of positively staining cells that represented some 1-3% of the population of cells within the human EBs, as opposed to less than 1% in the mouse ES (Efrat, 1998; Shamblott et al., 2001). Although this appears to occur spontaneously, it should be noted that the differentiation medium was supplemented with bFGF. Recently, Hart et al. (Hart et al., 2000) have suggested that FGF signaling may be involved in β-cell maturation, terminal differentiation and post-natal expansion. Tissue engineering estimates indicate that this is already a sufficient basis for enrichment protocols, based on the strategy described in Klug et al (Klug et al., 1996). Of importance, the finding that a subset of about 60% of the EBs contain insulin while the remainder do not, suggests that it may be useful to begin by selecting the subset of EBs with the highest percentage of insulin expressing cells as the source material for subsequent enrichment. Selecting this subset of EBs without sacrificing them, can be achieved using hES stably overexpressing a vital reporter marker driven by the insulin promoter. As exemplified herein below, additional protocols to enhance the starting number of β-cells in the mixed population of cells within the EBs can improve the yield from enrichment strategies.

The desired gene, the expression of which may for example confer growth advantage or increased efficiency of selection to the cells of the invention, is transferred to a cell culture. Numerous techniques are known in the art for the introduction of foreign genes into cells including electroporation, lipofection, microinjection, infection with a viral or bacteriophage vector containing are gene sequences, cell fusion, chromosome-mediated gene transfer, microcell-mediated gene transfer, calcium phosphate mediated transfection, spheroplast fusion, etc. (see e.g., Loeffler et al., 1993; Cohen et al., 1993; Cline, 1985). These methods may be used in accordance with the present invention, provided that the necessary developmental and physiological functions of the recipient cells are not disrupted. The methods should also provide a stable transfer of the gene to the ES cell, so that the gene is expressible by the cell and preferably heritable and expressible by its cell progeny. Usually, the method of transfer includes the transfer of a selectable marker to the cells. The cells are then placed under selection to isolate those cells that have taken up and are expressing the transferred gene.

### HI. Pharmacology

The ES cells of the present invention can be used to derive cells for therapy to treat an abnormal condition. The subject into which the insulin-producing cells derived from embryonic stem cells are introduced, or from which the pluripotent embryonic stem cells can be derived, is preferably an animal, including but not limited to animals such as cows, pigs, horses, chickens, cats, dogs, etc., and is preferably a mammal, and most preferably human. Preferably, the embryonic stem cell is originally derived from the subject to which it is administered, i.e., the transplant is autologous. For example, derivatives of human ES cells could be used as a source for insulin-producing β-cell replacement cell therapy.

The invention provides methods of treatment by administration to a subject of a pharmaceutical (therapeutic) composition comprising a therapeutically effective amount of a cell, preferably an insulin-producing cell derived from embryonic stem cell. Such a cell envisioned for therapeutic use is referred to hereinafter as a “Therapeutic” or “Therapeutic of the invention”. In a preferred aspect, the Therapeutic is substantially purified. The subject is preferably a mammal, and most preferably human.

The Therapeutic of the invention can be administered to a patient for the treatment of disease or injury by any method known in the art which is appropriate for the type of cells being transplanted and the transplant site. The cells can be transplanted intravenously, provided that they will be able to locate to the organ of interest In a specific embodiment, it may be desirable to administer the Therapeutics of the invention locally to the area in need of treatment. This may be achieved by, for example, and not by way of limitation, local infusion during surgery, topical application, e.g. transplanted directly into the pancreas at the site of implantation, by injection, by means of a catheter, or by means of an implant, said implant being of a porous, non-porous, or gelatinous material, including membranes, such as sialastic membranes, or fibers.

The present invention provides pharmaceutical compositions. Such compositions comprise a therapeutically effective amount of a Therapeutic, and a pharmaceutically acceptable carrier or excipient. Such a carrier includes but is not limited to saline, buffered saline, dextrose, water, glycerol, ethanol, and combinations thereof. The carrier and composition can be sterile. The formulation should suit the mode of administration.

The composition, if desired, can also contain minor amounts of wetting or emulsifying agents, or pH buffering agents. The composition can be a liquid solution, suspension, or emulsion.

In a preferred embodiment, the composition is formulated in accordance with routine procedures as a pharmaceutical composition adapted for intravenous administration or ectopic administration, preferably into pancreatic tissue of human beings. Where necessary, the composition may also include a solubilizing agent and a local anesthetic such as lignocaine to ease pain at the site of the administration.

Exemplary methods which can be modified for cell replacement therapy also termed herein cell transplantation are given by Lindvall et al., (Lindvall et al., 1989 and 1990).

The amount of the therapeutic composition of the invention which will be effective in the treatment of a particular disorder or condition will depend on the nature of the disorder or condition, and can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances.

### IV. Advantages of the Invention

As disclosed herein, the complex differentiation pattern of hES cells includes a subset of cells which have many characteristics of β-cell function, including proinsulin and/or insulin production and insulin release, as well as the expression of other β-cell markers. This finding is a necessary prerequisite for strategies based on cell enrichment from hES cells as a source of cell replacement in type 1 diabetes mellitus. Furthermore, the number of EBs with a surprisingly high percentage of such cells, is encouraging in terms of the potential prospects for successful use of insulin-producing human embryonic stem cells for cell replacement therapy.

In conclusion, we now disclose for the first time transgenic pluripotent undifferentiated hESc clones, transgenic for a reporter driven by a cell-specific promoter. This approach is useful for examination and monitoring of insulin producing cell differentiation. The present invention for the first time presents successful isolation and enrichment of insulin-producing cells derived form hESC, useful for cell transplantation therapy.

It will be appreciated by persons skilled in the art that the present invention is not limited by what has been particularly shown and described herein. Rather the scope of the invention is defined by the claims which follow.

### EXAMPLES

### Example 1

### Research Design and Methods

a. Tissue Culture

Large stocks of primary mouse embryonic fibroblasts (MEFs) were prepared as described by Robertson (Robertson et al., 1995), and stored in liquid nitrogen. After each thaw, cells were used for only 3-5 passages.

The hES H9 cells were maintained in the undifferentiated state by propagation in culture on a feeder layer of MEFs that have been mitotically inactivated by γ-irradiation with 35 Gy and plated on gelatin coated six-well plates. Cells were grown in knockout DMEM (GIBCO/BRL, Grand Island, N.Y.) supplemented with 20% serum replacement (GIBCO/BRL), 1% nonessential amino acids (GIBCO/BRL), 0.1 mM 2-mercaptoethanol (GIBCO/BRL), 1 mM glutamine (Biological Industries, Ashrat, Israel), 4 ng/ml human recombinant basic fibroblast growth factor (hrbFGF, PeproTech Inc, Rocky Hill, N.J.). Cultures were go in 5% CO2, 95% humidity and were routinely passaged every 4-5 days after disaggregation with 0.1% collagenase IV (Gibco/BRL).

b. Induction of Differentiation in hES Cells.

Methods for the induction of differentiation in mouse ES were applied herein for the induction of hES differentiation (Robertson et al., 1995; Keller, 1995). In brief, about 10⁷ undifferentiated hES cells were disaggregated and cultured in suspension in 100 mm bacterial grade petri dishes (Greiner, Frickenhausen, Germany), which results in induction of synchronous differentiation, characterized by initial formation of small aggregates, followed by the acquisition of the configuration of embryoid bodies (EBs) (Itskovitz-Eldor et al., 2000). Alternatively, hES colonies were left unpassaged until confluence (about 10 days), and then were replated on gelatinized six-well tissue culture plates in the absence of a feeder layer. The cells spontaneously differentiated to an array of cell phenotypes. The growth media that were used in differentiation were as described above.

C. Histological Analysis

EBs were collected at indicated intervals, washed three times with ice cold PBS, fixed overnight in 10% neutral buffered formalin, dehydrated in graduated alcohol (70-100%) and embedded in paraffin. For general histomorphology 5 μm sections were stained with hematoxylin/eosin.

d. Immunohistochemistry

Deparaffinized 5 μm sections were incubated for 90 minutes at room temperature with the primary antibody: polyclonal guinea pig anti-swine insulin, 1:100 dilution (Dako), followed by incubation with goat anti rabbit biotinylated secondary antibody. Detection was accomplished using streptavidin-peroxidase conjugate and AEC (or DAB) as a substrate (Histostain-SP kit, Zymed Lab Inc., CA). Counterstaining was carried out with hematoxylin. Non-immune serum was used as a negative control, and normal human pancreas paraffin sections were used as positive controls.

e. Morphometric Studies.

To estimate the relative percentage of cells staining positively by immunohisochemistry, morphometric measurements were conducted as previously described (Green et al., 2000).

f. Insulin Detection Assay

For adherent cells: MEFs, undifferentiated hES cells, cells which had differentiated spontaneously in vitro for more than 20 days were grown in 6 well plates. Cells were washed three times with serum free medium containing 25 mM glucose, and incubated in 3 ml serum free medium for two hours. For suspended EBs, experiments were performed in 50 mm bacterial grade petri dishes. Sixty to seventy EBs per plate were exposed to 3 ml serum free medium containing either 5.5 or 25 mM glucose. Subsequently conditioned media were collected and insulin levels were measured using a Microparticle Enzyme Immunoassay MEIA (Abbott AXSYM® system Insulin kit code B2D010) which detects human insulin, with no crossreactivaty to proinsulin or C-peptide. Data are represented as mean±s.e.m.

c. RT-PCR

Total RNA was isolated from undifferentiated hES cells, and from in vitro differentiated hES cells growing either as EBs or as high-density cultures at various stages of differentiation.

cDNA was synthesized from 7 μg total RNA using Moloney murine leukemia virus (M-MuLV) reverse transcriptase (Promega) in 1×transcription buffer containing 0.5 μmol/l oligo dT₍₁₂₋₁₈₎ (GIBCO/BRL) and 400 μmol/l dNTPs. Aliquots of cDNA were diluted 1:5 for IPF1/PDX1, neurogenin 3 (Ngn3), octamer-binding transcription factor (Oct4), Glut-1 and Glut-2, or 1:2 for insulin and islet specific glucokinase (GK). Subsequent PCR reactions were carried as follows: 2.5 μl (for IPF1/PDX1, Ngn3, Oct4) or 5 μl cDNA (for others), 1×PCR buffer, 400 μmol/l dNTPs, 100 ng of each primer pair and 1 U Taq polymerase. After initial hot start for 5 min, amplification continued with 28 cycles for β-actin, 31 cycles for Glut-1, 40 cycles for Glut-2, 38 cycles for glucokinase, 36 cycles for insulin, 37 cycles for Oct4, 35 cycles for IFP1/PDX1 and Ngn3. Denaturation steps were at 94° C. for 1 min, and annealing at 58, 52, 50, 67, 62, 55, 52 and 60° C., respectively, for 1 min, and extension at 72° C. for 1 min, and a final polymerization for 10 min. The amplified products were separated on 1.5% agarose gels. Each PCR reaction was performed in duplicate and under linear conditions. The forward and reverse primer sequences used for determination of human insulin, IPF1/PDX1, Ngn3 and β-actin were as follows: hIns: 5′-GCC TTT GTG AAC CAA CAC CTG-3′, 5′ GTT GCA GTA GTT CTC CAG CTG-3′ (261 bp fragment); IPF1: 5′-CCC ATG GAT GAA GTC TAC C-3′, 5′-GTC CTC CTC CTT TTT CCA C (262 bp fragment); Ngn3: 5′ CTC GAG GGT AGA AAG GAT GAC GCC TC-3′, 5′-ACG CGT GAA TGG GAT TAT GGG GTG GTG- 3′ (948 bp fragment); β-Actin: 5′-CAT CGT GGG CCG CTC TAG GCA C-3′, 5′-CCG GCC AGC CAA GTC CAG GAC GG-3′ (508 bp fragment), respectively. The primer sequences used for determination of Glut-1, Glut-2, GK and Oct4 were as previously described (Seino et al., 1993; Koranyi et al., 1992; van Eijk et al., 1999), the amplified fragments being 310, 398, 380 and 320 bps, respectively.

h. Statistics

Results are expressed as mean±s.e.m, and comparisons conducted using the unpaired Student's t-test.

### Example 2

### Insulin-producing Cells Derived Form Spontaneous Differentiation of H9 Line of Human ES Cells

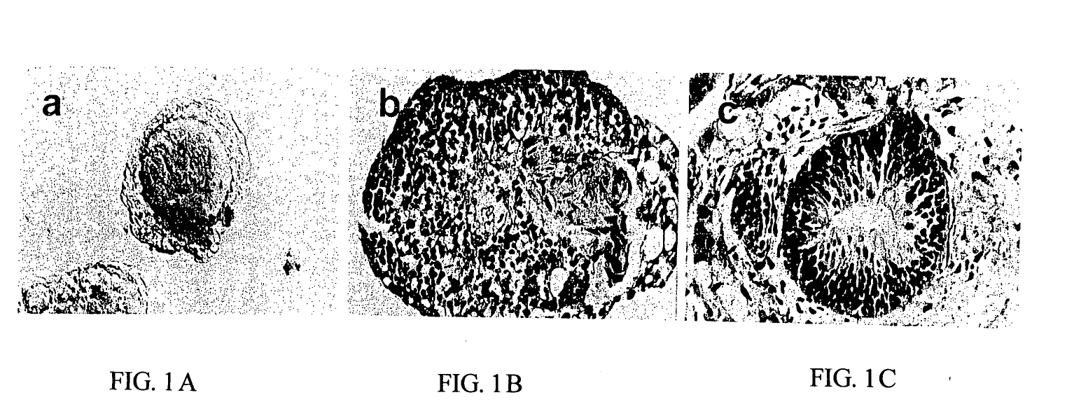

According to one currently preferred embodiment, the H9 line of hES cells were used. These cells grow as homogeneous and undifferentiated colonies when they are propagated on a feeder layer of MEFs. Accordingly, spontaneous in vitro differentiation of H9 cells was investigated following removal of cells from the MEF feeder layer, using two different model systems. Cells grown under adherent conditions in tissue culture plates, in the absence of MEFs displayed a pleiotropic pattern with numerous morphologies, including neuronal-like, muscle-like, or glandular-like structures (data not shown). In contrast, in vitro differentiation in suspension culture, resulted in a more consistent pattern with the formation of discrete EBs. One day after transfer to bacterial-grade petri dishes, cells failed to adhere and formed small aggregates. Following three days under these conditions, EBs acquired a simple structure with primitive endodermal layers surrounding inner cells (FIG. 1A-B), and then continued to grow in size and developed a more cystic structure (400-700 μm). These are similar to the morphologies reported for mouse EBs (Abe, 1996). Subsequent studies were carried out using immunohistochernistry (IHC) to determine the spatial and temporal pattern and to obtain an estimate of the relative density of cells in suspension cultured EBs with insulin producing capability. Hematoxylin and eosin staining of paraffin embedded sections was used to provide the overall histological morphology of EBs. Organization of EBs started as early as day three following removal from MEFs and transfer to suspension culture. With progressive days in suspension culture, more complex structures became evident, such as epithelial or endothelial-like cells lining hollow structures or cysts (FIG. 1C).

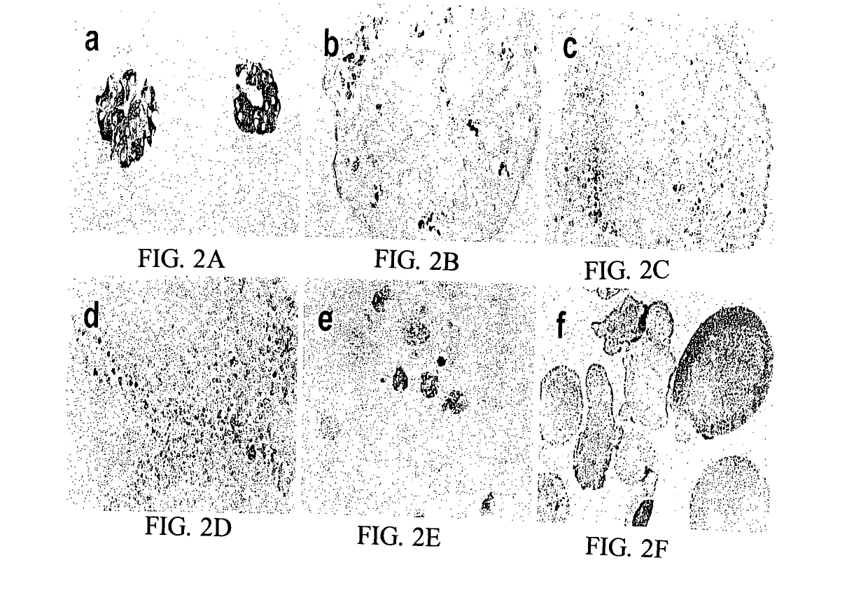

For monitoring EB development, EBs were collected every three days until day 19. Immunohistochemistry, using anti-insulin antibody, revealed cells expressing insulin as early as fourteen days of differentiation, with progressive increase in number through to day 19 (FIG. 2A-F). Insulin expressing cells were found either scattered throughout the EBs or organized into small clusters (FIG. 2A-F). Among EBs which stained positively for insulin (60-70%), on average 1-3% of cells were positively staining at maximum density. The remaining 30-40% of EBs were negative for insulin staining.

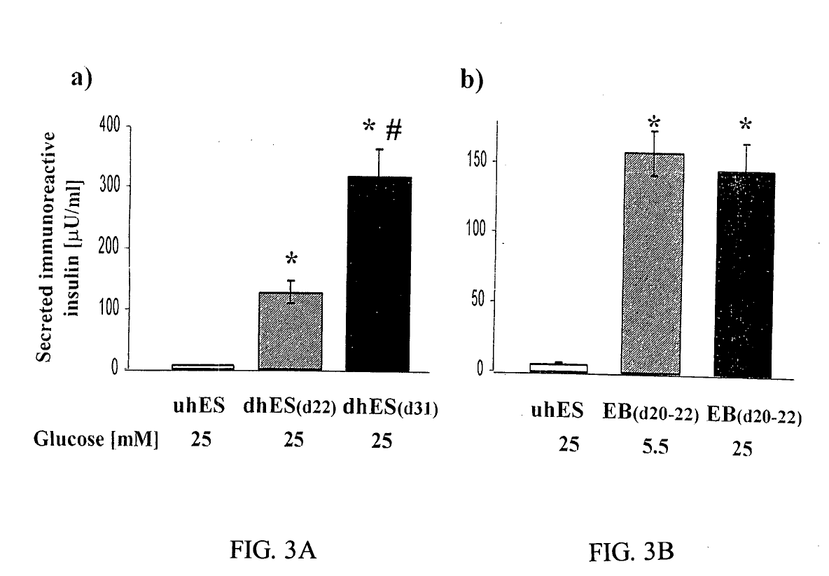

In order to characterize these insulin-containing cells, which are interspersed among the mixed population of spontaneously differentiating adherent hES, insulin elaborated into the medium was measured by enzyme immunoassay in undifferentiated hES (uhES), differentiated hES (dhES), and MEF cells. Growth medium contained serum replacement and 25 mM glucose, which is essential for hES viability. Insulin secretion was measured under both culture conditions of adherent cells (FIG. 3A), and from EBs (FIG. 3B). In adherent cells, insignificant immunoreactive insulin could be detected in serum-free media harvested from undifferentiated hES (5.6±0.6 μU/ml, n=6), and none from a feeder layer without overlying hES (data not shown). However, in serum-free media harvested after 22 and 31 days of differentiation, insulin concentrations were as follows: 126.2±17.7 (n=12) and 315.9±47 μU/ml (n=7), respectively (FIG. 3A). Insulin concentrations in dhES cell were significantly higher than in uhES cells (P<0.0001). In dhES cells, Insulin secretion after 31 days of differentiation were significantly higher than after 22 days (P=0.0004). Similarly, as shown in FIG. 3B, insulin release was significantly higher from 20-22 day EBs (60-70 EBs per each experiment) as compared to uhES (P<0.000).

In order to assess EB insulin responsiveness to different glucose concentrations. EBs containing culture dishes were acutely exposed to either 5.5 mM or 25 mM of glucose for two hours. These acute changes in medium glucose did not elicit any significant differences in elaborated insulin concentrations between the two groups (158±16 μU/ml, n=6 vs. 146.2±22.1 μU/ml, n=6, respectively, FIG. 3B).

### Example 3

### Expression of β-cell Related Genes in H9 bES Cells

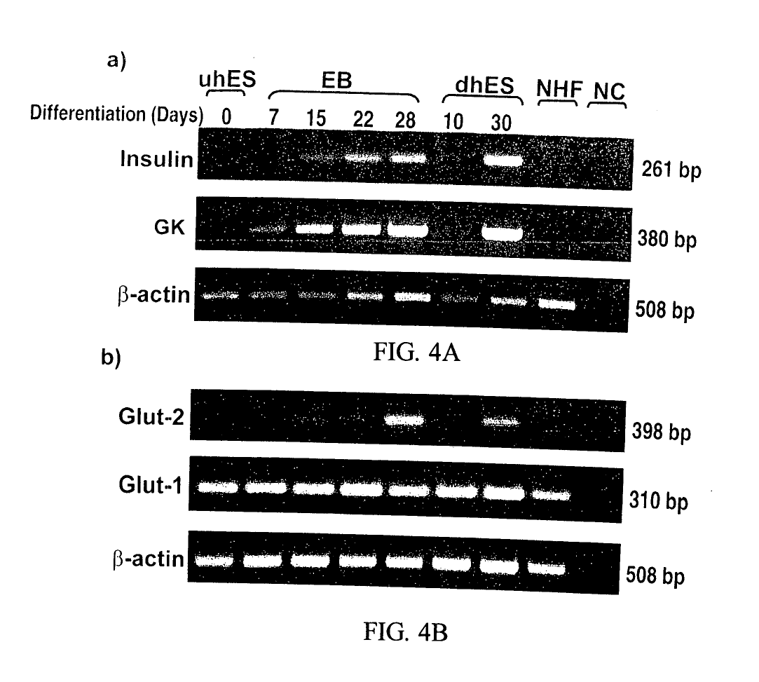

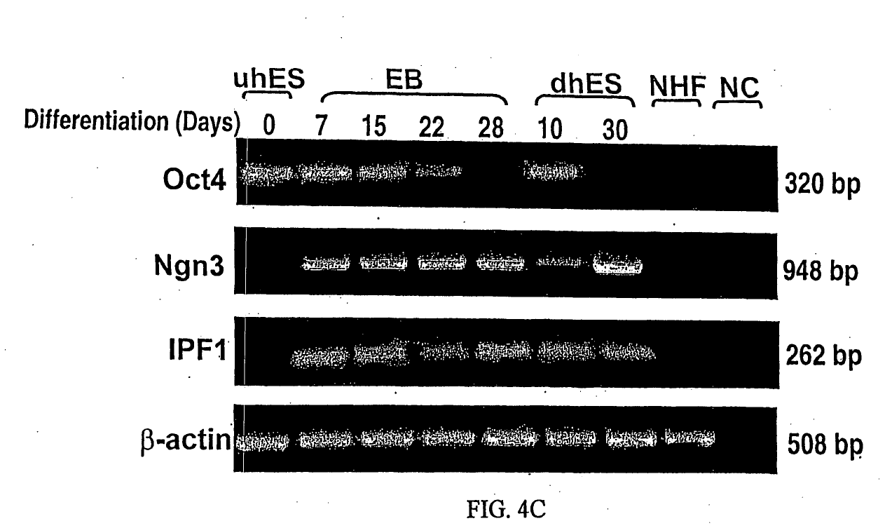

The foregoing example prompted us to examine the expression of other β-cell related genes using RT-PCR analysis of total RNA extracted from undifferentiated and differentiated hES cells. As shown in FIG. 4, insulin mRNA was detected in differentiated cells but not undifferentiated hES. In parallel, islet glucokinase (GK; FIG. 4A) and Glut-2 (FIG. 4B) genes were also identified following but not prior to differentiation. Similar results were obtained using either EBs or high-density adherent culture conditions. On the other hand, the Glut-1 isotype, a constitutive glucose transporter, was widely expressed in all forms of hES, as well as in human fibroblasts (FIG. 4B). Expression of three transcription factors: Oct4, Ngn3 and IPF1, was examined (FIG. 4C). As expected, mRNA expression of Oct4, a marker of the pluripotent state (Yeom et al., 1996; Niwa et al., 2000), was detected in undifferentiated hES, but decreased progressively during the subsequent three weeks of differentiation (FIG. 4C). We also demonstrated that differentiating hES cells express IPF1/PDX1 and Ngn3 transcription factors (FIG. 4C), that together have been shown to regulate pancreatic and endocrine cell differentiation (Edlund 1998; Apelqvist et al., 1999; Schwitzgebel et al., 2000; Herrera, 2000).

The expression of β-actin, in parallel to the expression of the β-cell related gene served as an internal control for RNA loading and blotting differences between lanes.

### Example 4

### Insulin Producing Cells Derived from hES Cells Overexressing Human Telomerase Reverse Transcriptase (hTERT).

Since pluripotent hES cells are capable of differentiating into many cell types, they or their derivatives can be used for research and medical applications, including cellular transplantation. A major objective of this invention is to modulate the differentiation of hES cells so that a desired population of precursors or fully differentiated cells can be obtained.

We have already provided evidence that a surprisingly high number of insulin producing β-cells appear during the differentiation of hES in culture, under the conditions that we have utilized (see Example 2). This observation is a prerequisite for experimental strategies based upon the enrichment of spontaneously appearing 62 -cells or precursors for tissue engineering and cell replacement therapy in the treatment of diabetes mellitus.

However, it is conceivable that enrichment and propagation of large number of such differentiated cells will suffer the disadvantage of low proliferation capacity and limited life span. To overcome this limitation, agents which may confer growth advantage (e.g. telomerase) may be utilized. Telomerase in particular may be used to maintain telomere length and integrity and thereby extend the proliferation capacity of the cells. In those instances where ectopic over-expression of hTERT does not adversely influence the differentiation pattern, it is possible to generate a fully diferentiated desired lineage of telomerase positive cells.

### Experimental Protocol

a. Ectopic Expression of hTERT in Undifferentiated hES Cells.

hTERT over-expressing hES cells are allowed to differentiate under the same conditions chosen for selection and enrichment for production of insulin producing cells (combination of factors such as insulin+transferrin+sodium selenite (ITS), glucose, nicotinamide, keratinocyte growth factor (KGF), fibroblast growth factor (FGF), vascular endothelial growth factor (VEGF), epidermal growth factor (EGF), nerve growth factor (NGF), activin, β-cellulin, in the culture medium).

According to one currently most preferred embodiment of the invention it is now possible to significantly enhance the yield of insulin producing cells using the combination of factors known as ITS, namely insulin, transferrin and selenite. One preferred salt is sodium selenite but other salts containing selenium may also be utilized.

We use the strategy of genetic modification of human ES cells using a cell specific promoter-neo^{r} transgene in a way that permits the generation of homogeneous insulin producing cells, by utilizing an appropriate (e.g. bicistronic) expression vector that contains an attenuated internal ribosome entry site (IRES). This vector is constructed so that hTERT coding sequence is driven by PDX-1, a cell specific gene promoter that becomes active in β-cells progenitors at the very early stages of β-cells differentiation pathway. Alternative promoters may substitute PDX-1. The hTERT coding region is constructed to reside in a single cassette with an IRES and hygromycin or other antibiotic resistance selection marker. In addition this vector carries a neomycin (or other antibiotic) selection marker under the control of a constitutive promoter, including for example PGK promoter or another mammalian promoter. The construct is transfected into undifferentiated hES cells and neomycin resistant clones are selected. In the subsequent step, these clones are allowed to differentiate and hygromycin resistant clones are selected. Virtually, all the transfected cells, which express the selection marker, are expected to express telomerase and most likely differentiate into the insulin producing cell lineage that has the ability to activate the PDX-1 cell specific promoter at the very early stages of this differentiation pathway.

In order to prevent the toxic effect of high glucose on the PDX-1 promoter due to the high glucose concentration in the growing medium of hES cells, antioxidant reagents, including but not limited to Nicotinamide, L-ribose, N-acetyl-cysteine, and others antioxidants are added to the culture medium.

b. Criteria for Selection of Clones

The resultant clones are examined for advantageous properties and/or lack of deleterious properties, as follows:

1. Examination at the undifferentiated state for expression of the Neor gene using RT-PCR to avoid proceeding with the selection procedure with false positive clones.

2. Examination of the resultant clones following differentiation and hygromycin selection for expression of exogenous hTERT or other proliferation (extended life-span) promoting gene using northern blot analysis or RT-PCR, for Telomerase activity using TRAP assay and for telomere length using TRF assay. The above are monitored at multiple time intervals following differentiation. As control, normal hES that went through the same process are used.

3. Examination of the expression profile of factors involved in β-cells differentiation and maturation using quantitative RT-PCR. The expression of the following genes are monitored: Insulin, PDX-1, Nkx6.1, Nkx2.2, Glut-2, Pax-6, BETA2, Ngn3, Islet-1, Pax4, Hlxb-9, GK, Nestin, Prohormone convertase (PC) 1 and 2, glucagons, GAD65. The pattern of expression is compared with control cells. A microchip for expression profiling is used to facilitate the screen, if needed.

4. Examination of specific enzyme profile using enzymatic assays for Glut2, GK, PC.

5. Quality control of insulin producing cells derived as above from hES cells will be carried out using the following parameters: ultrastructural characterization using electron microscope, insulin secretion, insulin processing, electrophysiological profile, metabolic profile.

6. Compare β-cells developmental gene expression profile in the clones versus normal cells using microarray chips.

7. Immunohistochemistry or western blot analysis using specific antibodies for β-cells specific markers.

8. Lack of tumorigenic properties are examined according to any of the criteria as are known in the art, including the ability to generate tumors in nude mice, the ability to grow on soft agar (focus formation), and presence of a normal cell karyotype.

C. Stable Transfection of hTERT Into Fully Differentiated Cell Lineage

Following enrichment and characterization of a fully differentiated cell lineage, e.g. insulin producing β-cells, hTERT gene coding sequence driven by a powerful promoter such as the β-actin gene promoter or PGK gene promoter, are stably transfected with a selection marker, to overcome the disadvantage of low proliferation capacity and limited life span. This strategy is to promote proliferation of insulin producing β-cells that might replace the necessity of donors for islet transplantation The final clonal population is examined for all the criteria mentioned above. In parallel, this clonal population is examined for telomerase activity, telomere length, extension of life span and lack of tumorigenic properties as outlined above.

d. Ectopic Expression of hTERT and/or Growth Enhancing Gene in Undifferentiated hES Cells in a Combination with Cre-recombinase Gene

For establishing a large population of insulin producing cells for cell therapy, eventually it is necessary to overcome the problems of limited life span and proliferation capacity of mature finally differentiated β-cells. As described above, to overcome this limitation telomerase activity may be utilized to maintain telomere length and integrity and thereby extend the proliferation capacity of the cells. Alternatively, a growth-enhancing gene can be inserted into the hES cells similar to the strategy described above. In brief, the coding region of a growth enhancing gene such as SV40 large T-antigen, under the control of PDX-1 promoter is constructed in a single cassette with an IRES and hygromycin selection marker in a bicistronic vector that carries also neomycin resistant gene under the control of a constitutive promoter of the β-actin gene or the PGK gene. Progenitor cell populations are allowed to expand for several orders of magnitude following induction of terminal differentiation into mature β-cells. These mature cells are examined using the criteria described above with an emphasis on criterion number 8, namely the lack of tumorigenicity.

For the purpose of cell transplantation DNA sequences with the potential to develop tumorigenicity are removed from the cells prior to transplantation. hTERT gene is not an oncogene, but its reactivation occurs in 85-95% of the cancers. In addition most of the growth enhancing genes have the capacity to confer malignant characteristic of the cells.

For this purpose we utilize the Cre recombinase-loxp (Cre-loxP) system to confine the inactivation and elimination of the target sequences in a cell specific manner. Cre-recombinase functions as a site specific recombinase, splicing DNA sequences between specific 34-bp sequences known as loxP sites. The unique property of the insulin gene to be expressed only in pancreatic β-cells, allows using the β-cells specilic insylin promoter for Cre recombinase expression to inactivate and eliminate genes in fully matured β-cells. The protocol uses stably transfected hES with hTERT or a growth enhancing gene coding sequences under the control of β-cells progenitor cell specific promoter PDX-1, as described above, in a single cassette with an IRES and hygromycin or other antibiotic selection marker. lacZ gene coding sequence inserted independently downstream of the hygromycin gene remains unexpressed in the progenitor cells as long as Cre reconibinase is not activated. The hTERT-IRES-Hygromycin sequences are flanked by loxp (floxed) to allow excision of this sequence following expression of Cre recombinase in the cells. Upon activation of insulin promoter and thereby the Cre recombinase gene in fully matured β-cells, this sequences is floxed out allowing the lacZ coding sequence to be expressed under the control of the PDX-1 promoter. The expression of the lacZ gene depends on the expression of the Cre recombinase, therefore a time course study is essential to determine the optimal time required for maximum recombination to excise the target gene in β-cells that results in expression of the lacZ gene.

Measurement of lacZ enzyme activity is carried out using a fluorogenic substrate, which is hydrolyzed and retained intracellularly. The advantage of this system is that lacZ serves both as a reporter gene to quantitate recombination efficiency and as a selectable marker for the fluorescence-activated sorting of cells based on their lacZ expression level.

Another variant of this approach, utilizes the Cre-recombinase driven by the insulin promoter in cells in which the thymidine kinase gene has been stably transfected in between loxp sites. Cell toxicity is induced by addition of ganciclovir, and only those cells in which the TK is specifically floxed out (cell specific promoter driving Cre-recombinase) survive the ganciclovir treatment.

It will be appreciated by the artisan in the field to which this invention pertains that all of the foregoing protocols may be generalized to other cell-specific promoter based systems.

e. Glucose Responsiveness

For glucose responsiveness our protocols utilize pre-incubations in media with varying glucose concentrations, with or without the addition of antioxidants. As a measure of appropriate incubation glucose concentrations for longer-terms culture, PDX-1 binding by EMSA is monitored.

Of importance, the finding that a subset of about 60% of the EBs contain insulin while the remainder do not, suggests that it may be useful to begin by selecting the subset of EBs with the highest percentage of insulin expressing cells as the source material for subsequent enrichment Selecting this subset of EBs without sacrificing them, can be achieved using hES stably overexpressing a vital reporter marker driven by the insulin promoter.

Alternatively, the EBs population will be seeded in 96 well plates; one EB per well and insulin responsiveness will be demonstrated by measuring insulin secretion to the medium.

### Example 5

### Establishment of hES Cells Clones Stably Transfected with Insulin Promoter Driving Enhanced Green Fluorescent Protein (EGFP) Reporter Gene

a. Construction of Plasmids Containing Human Insulin Promoter (pIns)

A DNA fragment containing 327 bp of the 5′-flanking region and 30 bp of exon 1 of the human insulin gene was amplified using the polymerase chain reaction (PCR). The following oligodeoxynucleotides were used as primers:

5′-GCG GAG CTC TCT CCT GGT CTA ATG TGG AA-3′
5′-GCG CTC GAG CTC TTC TGA TGC AGC CTG TC-3′

The sequence of the human insulin regulatory fragment in the new construct was verified by sequencing using the ABI PRISM® Big Dye™ Terminator Cycle Sequencing Ready Reaction Kit (Applied Biosystem).

This fragment was subcloned into pBluescripte® II KS (Stratagene) using SacI and XhoI restriction enzyme sites. The pBluescrip containing the pIns was digested by SacI and KpnI and the resulting fragment was inserted in the correct orientation in the MCS of pEGFP-1 (Clontech). This vector carries also a neomycin resistance gene drived by a constitutive SV40 promoter. This feature is important in further studies for selection pressure in stable transfection experiments.

pEGFP-1 backbone was used for the second construct where the insulin core promoter driving hygromycin resistance gene fused upstream of EGFP. The advantage of using this reporter gene is that it provides the convenience of drug resistance selection marker with the ability to identify EGFP positive cells.

Activity of the insulin promoter in both expression vectors was tested using transient transfection of Hamster insulinoma tumor cells (HIT), which revealed EGFP fluorescence in transfected cells.

b. Stable Transfection

H9 hES cells were cultured in six well plates and transfected after 24 hours using 3 μg of plasmid DNA and 6 μl of the non-liposomal formulation FuGENET™ transfection reagent (Boehringer Mannheim).

Positive clones were isolated and propagated in the presence of 100 μg/ml of G418 in the growing medium and characterized for expressing the Neo resistance gene using Northern Blot analysis.

c. Generation of Teratomas from Transfected Clones

Undifferentiated cells from stable clones were injected intramuscularly into the hind limb of nude mice (˜5×10⁶ cells per site). After 70-90 days teratomas were harvested, fixed, embedded in paraffin and 5 μm sections were prepared. For histomorphology, sections were stained with hematoxylin/eosin. For Immunohistochemistry analysis, deparaffinized sections were incubated with anti-insulin antibody as described before (Assady et al., 2001).

d. Establishment of Insulin Positive Cell Population

Six positive clones stably transfected with the insulin promoter-EGFP reporter, and one clone stably transfected with the insulin promoter-HygEGFP reporter were established.

Transfected undifferentiated cells maintained normal morphology, as compared to wild type H9 cells.

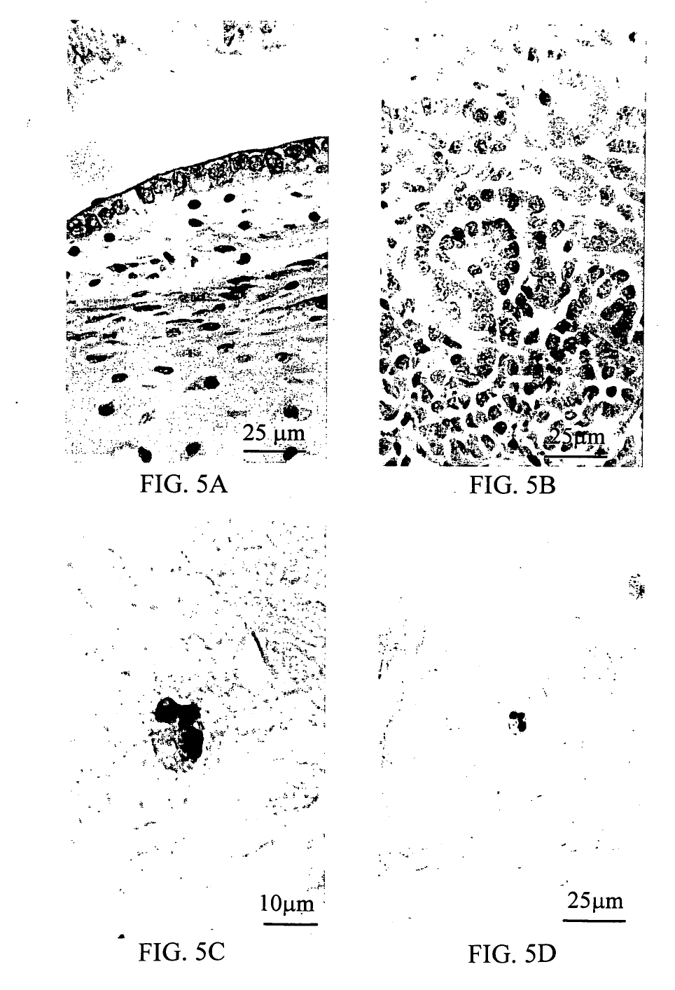

All new clones generated cell aggregates (embryoid bodies) when grown in suspension cultures, and formed advanced derivatives of all three embryonic germ layers following injection into nude mice (FIG. 5A-B). Small clusters, rare but evident, of insulin positive cells were observed by IHC (FIG. 5C-D), which may reflect the lack of developmental advantage for cells carrying the specific transgene under normoglycemic physiological state in vivo.

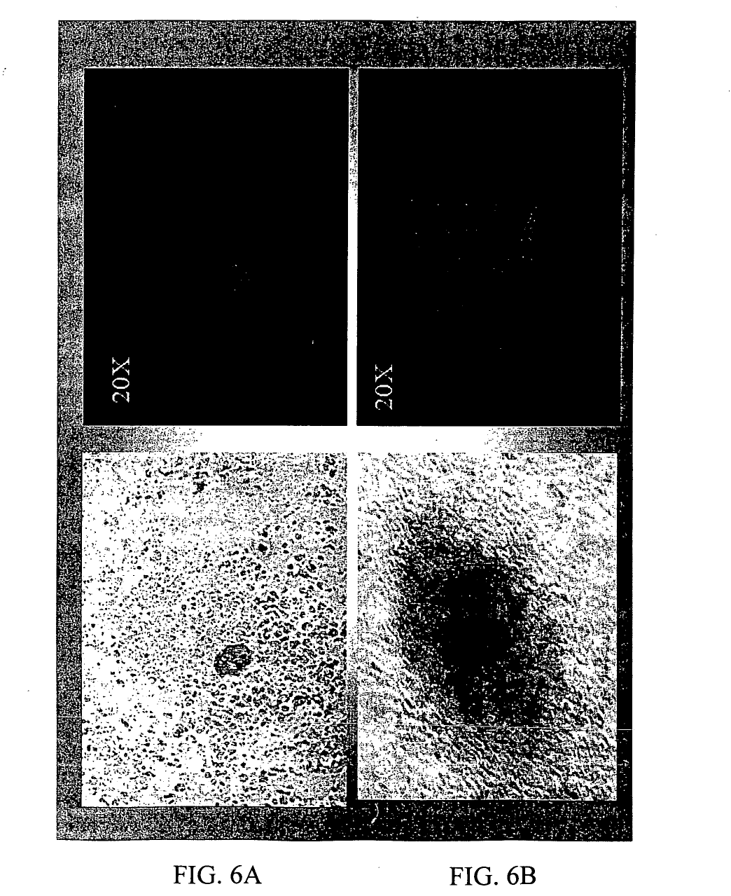

EGFP was negative in the undifferentiated state and became visible when grown under differentiation conditions (FIG. 6). Of note, among culture wells, there was a correlation between EGFP fluorescence as observed by microscopy and measured insulin secretion and content. For instance, the IIB6 clone which exhibited low fluorescence and low insulin content (FIG. 6A; 282 μU/ml) as compared with the IB3 clone (FIG. 6B; 1015 μU/ml), also secreted less insulin (43 μU/ml) than secreted by the IB3 clone (364.5 μU/ml). Morphologically, EGFP expressing cells were often small and grew in dense clusters. These cells were separated using fluorescence activated sorting, based on EGFP fluorescence, and molecular and physiological features are currently being examined.

e. Enrichment of Insulin Positive Cell Population.

Different protocols are compared. Preliminary results showed that growing hES as EBs for 15 days, followed by seeding on matrigel in the presence of ITS X1 (Sigma, or GIBCO) and FGF 4 ng/ml in the growth medium for the subsequent 4-5 weeks, yielded enrichment of 5% as estimated by FACS analysis in IB3 clone (protocol 1 and FIG. 6B).

Next, a modified version of a reproducible staged enrichment protocol for enrichment of β-like cells from mouse ES, recently published by Lumelsky et al, was adapted in combination with the tracking enrichment strategy, utilizing EGFP reporter gene (protocol 2). Briefly, the protocol consited of the following stages:

Stage I: pluripotent undifferentiated cells of transfected clones were grown on a feeder layer of MEFs in 10 cm tissue culture dishes (Nunc) as described previously (Assady et al., 2001), for 4-5 days.

Stage II: following disaggregation using collagenase type IV 0.1%, cells were transferred to suspension cultures, in the same growth medium but in the absence of hrbFGF, for 4-5 days.

Stage III: disaggregation of EBs by trypsin/EDTAX1 (GIBCO/DRL) for 5 minutes, following proliferation on fibronectin coated 6-well tissue culture dishes, for 7-10 days, in serum free DMEM/F12 medium containing fibronectin 1 μg/ml, and ITS x1.

Stage IV: cells were grown on laminin/polyornithine or laminin/polylysine coated dishes in serum free DMEM/F12 medium containing B27 supplement (GIBCO), N2 supplement (GIBCO), laminin 1 μg/ml, FGF 10 μg/ml, for 7-8 days.

Stage V: medium was replaced with medium containing nicotinamide 10 μg/ml, B27, N2 and laminin as in stage 4, for 7-10 days.

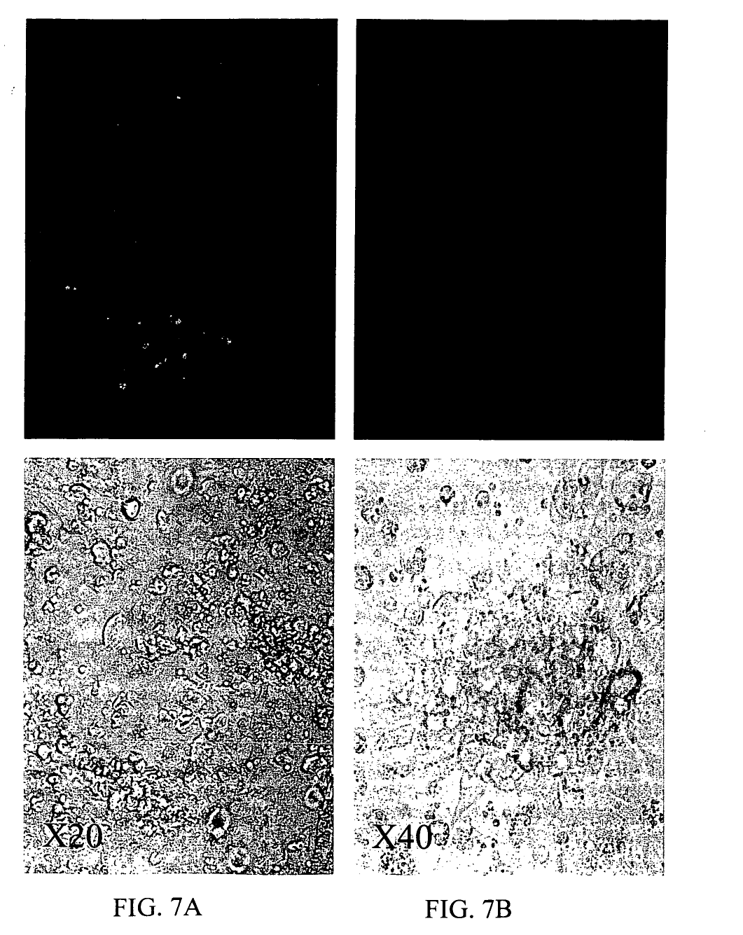

The enrichment protocol was repeated for eight times and a reproducible pattern of growth and differentiation was observed. An initial fluorescence signal was observed on day 4 and 5 of stage m as shown in FIG. 7A (day 4), where in adherent layers of cells, groups of fluorescent cells are observed scattered throughout the field in a pattern highly reminiscent of the positive insulin immuno-fluorescence which was shown as red staining in the Lumelsky report at the corresponding stage. By the end of stage IV or V, clusters of EGFP expressing cells, which are easily dislodged, were surrounded by neural-like projections (FIG. 7B; day 2).

Molecular and physiological features are currently being examined.

In conclusion, transgenic pluripotent undifferentiated hES cell clones transgenic for a reporter driven by a cell specific promoter were generated. This approach can be utilized to examine and monitor insulin-producing cell differentiation, and to subsequently isolate enriched populations of insulin-producing cells for possible future transplantation therapy.

The specific growth conditions described above, or components thereof, are those which constitute the basis of protocols for promoting the favorable growth of insulin producing cells from hES. It will clearly be recognized by the skilled artisan that it is possible to change many of the particulars of these protocols without departing from the essentials of the invention. All of the many possible variants may be considered optimization of parameters within the framework of the invention disclosed. The scope of the invention is defined by the claims which follow.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention Thus the expressions “means to . . . ” and “means for . . . ”, or any method step language, as may be found in the specification above and/or in the claims below, followed by a functional statement, are intended to define and cover whatever structural, physical, chemical or electrical element or structure, or whatever method step, which may now or in the future exist which carries out the recited function, whether or not precisely equivalent to the embodiment or embodiments disclosed in the specification above, i.e., other means or steps for carrying out the same functions can be used; and it is intended that such expressions be given their broadest interpretation.

REFERENCES

The Diabetes Control and Complication Trial Research Group. N Eng J Med 329:977-86 (1993)

Abe K Exp Cell Res 229:27-34 (1996)

Alarcón C, Serna J, Pérez-Villamil B, de Pablo F. FEBS Letters 436:361-6 (1998)

Amit M, Carpenter M K, Inokuma M S, Chiu C P, Harris C P, Waknitz N A, Itskovitz-Eldor J, Thomson J A. Dev Biol 227:271-8 (2000)

Apelqvist A, Li H, Sommer L, Beatus P, Anderson D J, Honjo T, Hrabe de Angelis M, Lendhal U, Edlund H, Nature 400:877-881 (1999)

Assady et al. Diabetes 50:1679-91 (2001)

Bonner-Weir S, Taneja M, Weir G C, Tatarkiewics K, Song K H, Sharmia A, O'Neil J J. Proc Natl Acad Sci USA 97:7999-8004 (2000)

Charollais A, Gjinovci A, Huarte J, Bauquis J, Nadal A, Martin F, Andreu E, Sánches-Andrés J, Calabrese A, Bosco D, Soria B, Wolheim C B, Herrera P L, Meda P. J Clin Invest 106:235-43 (2000)

Cheung A T, Dayanandan B, Lewis J T, Korbutt G S, Rajotte R V, Bryer-Ash M, Boylan M O, Wolfe M M, Kieffer T J. Scieince 290:1959-62 (2000)

Cline M J, Pharmac. Ther. 29:69-92 (1985)

Cohen et al., Meth. Enzymol. 217:618-44 (1993)

Edlund, H Diabetes 47:1817-23 (1998)

Edlund H: Pancreas. Curr Op Cell Biol 11:663-8 (1999)

Efrat S. Nat genet 17:249-250 (1997)

Efrat, S. Diabetologia 41:1401-9 (1998)

Efrat S. Ann N Y Acad Sci 875:286-93 (1999)

Ferber S, Halkin A, Cohen H, Ber I, Einav Y, Goldberg I, Barshack I, Seijffers R, Fioretto P, Steffes M W, Sutherland D E, Goetz F C, Mauer M. N Eng J Med 339:69-75 (1998)

Green J, Maor G. Kidney Int 57:2258-2267 (2000)

Halvorsen T L, Beattie G M, Lopez A D, Hayek A, Levine F. J Edocrinol 166:103-9 (2000)

Hart A W, Baeza N, Apelqvist A, Edlund H Nature 408:864-8 (2000) Herrera P L, Development 127:2317-22 (2000)

Itskovitz-Eldor J, Schuldiner M, Karsenti D, Eden A, Yanuka O, Amit M, Soreq H, Benvenisty N. Mol Med 6:88-95 (2000)

Jonkers F C, Jonas J C, Gilon P, Henquin J C. J Physiol 520:839-49 (1999)

Keller G M. Curr Op Cell Biol 7:862-9 (1995)

Kim S K, Hebrok M. Genes & Dev 15:111-27 (2001)

Klug M G, Soonpaa M H, Koh G Y, Field L J. J Clin Inv 98:216-24 (1996)

Kopolovic J, Kaiser N, Karasik A. Nat Med 6:568-572 (2000)

Koranyi L I, Tanizawa Y. Welling C M, Rabin D U, Permutt M A. Diabetes 41:807-11 (1992)

Lee H C, Kim S J, Kim K S, Shin H C, Yoon J W. Nature 408:483-488 (2000) Lindvall O, Rehncrona S, Brundin P, Gustavii B, Astedt B. Widner H, Lindholm T, Bjorklund A, Leenders K L, Rothwell J C et al., Arch. Neturol. 46:615-31 (1989)

Lindvall O., Brundin P, Widner H, Rehncrona S, Gustavii B, Frackowiak R, Leenders K L, Sawle G, Rothwell J C, Marsden C D et al., Science 247:574-7 (1990)

Ling Z, Pipeleers D G. J Clin Inv 98:2805-12 (1996)

Loeffler J P and Behr J P, Meth. Enzymol. 217:599-618 (1993)

Matschinsky F M, Glaser B, Magnuson M A. Diabetes 47:307-15 (1998)

Niwa H, Miyazaki J I, Smith A G, Nat genet 24:372-376 (2000)

Ramiya V K, Maraist M, Arfors K E, Schatz D A, Peck A B, Cornelius J G, Nat Med 6:278-82 (2000)

Reubinoff B E, Pera M Y, Fong C Y, Trounson A, Bongso A. Nat Biotech 18:399-404 (2000)

Robertson E: Teratocarcinomas and embryonic stem cells a practical approach. Practical approach series. IRL press. 71-112 (1987)

Samstein B, Platt J L. J Am Soc Nephrol 12:182-93 (2001)

Schuldiner M, Yanuka O, Itskovitz-Eldor J, Melton D A, Benvenisty N. Proc Natl Acad Sci USA 97:11307-12 (2000)

Schwitzgebel V M, Scheel D W, Conners J R, Kalamaras J, Lee J E, Anderson D J, Sussel L, Johnson J D, German M S, Development 127:3533-3542 (2000)

Seino, Y. et al. J Clin Endocrinol Metab 76:75-8 (1993)

Shamblott M J, Axelman J, Wang S, Bugo E M, Littlefield J T, Donovan P J, Blumenthal P D, Huggins G R, Gearhart J D. Proc Natl Acad Sci USA 95: 13726-31 (1998)

Shamblott M J, Axelman J, Littlefield J W, Blumenthal P D, Huggins G R, Cui Y, Cheng L, Gearhart J D. Proc Natl Acad Sci USA 98: 113-8 (2001)

Shapiro A M J, Lakley J R T, Ryan E A, Korbutt G S, Toth E, Warnock G L, Kneteman N M, Rajotte R V. N Eng J Med 343:230-8 (2000)

Shepherd P R, Kahn B B: Mechanism of disease. N Eng J Med 341:248-57 (1999)

Soria B, Andreu E, Berná G, Fuentes E, Gil A, León-Quinto T, Martin F, Montaya E, Nadal A, Reig J A, Ripoll C. Roche E, Sanches-Andrés J. Pflügers Arch-Eur J Physiol 440:1-18 (2000a)

Soria B, Roche E, Berná G, León-Quinto T, Reig J A, Martin F. Diabetes 49:157-161 (2000b)

Thomson J A, Itskovitz-Eldor J, Shapiro S S, Waknitz M A, Swiergiel J J Marshal V S, Jones J M. Science 282:1145-7(1998)

Tzukerman M, Shachaf C, Ravel Y, Braunstein I, Cohen-Barak O, Yalon-Hacohen M, Skorecki K L. Mol Biol Cell 11:4381-91(2000)

van Eijk M J, van Rooijen M A, Modina S, Scesi L, Folkers G, van Tol H T, Bevers N M, Fisher S R, Lewin H A, Rakacolli D, Galli C, de Vaureix C, Trounson A O, Mummery C L, Gandolfi F. Biol Reprod 60:1093-103 (1999)

Yeom Y I, Fuhrmann G, Ovitt C E, Brehm A, Kazuyuki O, Gross M, Hübner K, Schöler H R, Development 122:881-94 (1996)

1. A cell population comprising insulin-producing cells derived from human embryonic stem cells.

2. The cell population of claim 1 enriched for insulin-producing cells derived from human embryonic stem cells.

3. The cell population of claim 2 wherein the enrichment comprises treatment of the human embryonic stem cells with insulin, transferrin and selenite.

4. The cell population of claim 1 comprising selected insulin-producing cells derived from human embryonic stem cells.

5. The cell population of claim 1 comprising isolated insulin-producing cells derived from human embryonic stem cells.

6. The cell population of claim 1 comprising cloned insulin-producing cells derived from human embryonic stem cells.

7. A cell population comprising regulatable insulin-producing cells derived from human embryonic stem cells.

8. The cell population of claim 7 comprising glucose-responsive insulin-producing cells derived from human embryonic stem cells.

9. The cell population of claim 8 enriched for glucose-responsive insulin-producing cells derived from human embryonic stem cells.

10. The cell population of claim 9 wherein the enrichment comprises treatment with insulin, transferrin and selenite.

11. The cell population of claim 8 comprising selected glucose-responsive insulin-producing cells derived from human embryonic stem cells.

12. The cell population of claim 8 comprising isolated glucose responsive insulin-producing cells derived from human embryonic stem cells.

13. The cell population of claim 8 comprising cloned glucose-responsive insulin-producing cells derived from human embryonic stem cells.

14. The glucose responsive insulin-producing cells of claim 8 wherein said cells express at least one gene from the group of: insulin, islet glucokinase, Glut-2 glucose transporter, Glut-1 glucose transporter, insulin promoter factor1/pancreatic and duodenal homeobox gene 1 IFP1/PDX1 transcription factor, and Ngn3 transcription factor.

15. A cell population comprising stable insulin-producing cells derived from human embryonic stem cells.

16. The cell population of claim 15 comprising stable clonal insulin-producing cells derived from human embryonic stem cells.

17. The cell population of claim 15 comprising insulin-producing cells derived from human embryonic stem cells overexpressing hTERT.

18. The cell population of claim 15 comprising insulin-producing cells derived from human embryonic stem cells stably transfected with a construct comprising an insulin promoter.

19. The cell population of claim 18 comprising cloned insulin-producing cells derived from human embryonic stem cells stably transfected with an insulin promoter.

20. A clone of non-differentiated human stem cells stably transfected with a vector comprising the DNA coding sequence of human insulin promoter.

21. A cell population comprising pluripotent precursors of beta islet cells of the pancreas derived from human embryonic stem cells, stably transfected with an insulin promoter.

22. A cell population comprising committed precursors of beta islet cells of the pancreas derived from human embryonic stem cells.

23. The cells clone of claim 20, wherein a reporter gene is fused downstream of the insulin promoter sequence.

24. The cells clone of claim 20, wherein the expression of the reporter gene is regulated by the insulin-promoter gene.

25. The cells clone of claim 20, comprising insulin-producing cells.

26. A method for in vitro enrichment of insulin-producing cells derived from stem cells, comprising the steps of:
(i) culturing undifferentiated pluripotent stem cells in a chemically defined serum-free culture medium complemented with supplements selected from: serum replacement; nonessential amino acids; mercaptoethanol; glutamine; or fibroblast growth factor, and
(ii) disaggregating and transferring the adherent cell cultures from (i) to suspension culture in bacterial-grade petri dish; and
(iii) adding to the culture medium of the cells from (ii) supplements selected from the group consisting of: insulin; transferrin and sodium selenite (ITS); glucose; nicotinamide; keratinocyte growth factor; fibroblast growth factor; vascular endothelial growth factor; epidermal growth factor; nerve growth factor; activin; and β-cellulin.

27. The method in claim 26 comprising the following step:
(i) culturing undifferentiated pluripotent stem cells on a feeder layer in a chemically defined serum-free culture medium complemented with supplements selected from: serum replacement; nonessential amino acids; mercaptoethanol; glutamine; or filroblast growth factor; and
(ii) disaggregating and transferring the adherent cell cultures from (i) to suspension culture in bacterial-grade petri dish; and
(iii) culturing the cells in (ii) for 4-5 in a culture medium as in (i) in the absence of fibroblast growth factor; and
(iv) disaggregating and transferring the embryoid bodies formed in (iii) to fibronectin coated tissue culture dishes in serum-free medium;
(v) adding to the culture medium of (iv) supplements selected from the group consisting of: fibronectin; transferrin and sodium selenite (ITS); and
(vi) adding to the culture medium of (v) supplements selected from the group consisting of: B27 supplement (GIBCO); N2 supplement (GIBCO); laminin: and fibroblast growth factor; and
(vii) replacing the culture medium in (vi) with culture medium comprising supplements selected from the group consisting of: B27 supplement (GIBCO); N2 supplement (GIBCO); laminin; and nicotinamide.

28. In a method of cell replacement therapy, the improvement which comprises administering to a subject in need of such therapy insulin producing cells derived from human embryonic stem cells.

29. The method of claim 28 wherein the cells are transplanted into the subject's pancreas.

30. The method of claim 28 wherein the cells are transplanted into an ectopic site in the subject.

31. A method of treating a patient in need thereof with insulin producing cells derived from human embryonic stem cells comprising transplantation of a cell population comprising insulin producing cells derived from human embryonic stem cells.

32. The method of claim 31 comprising transplantation of a cell population comprising insulin producing cells derived from human embryonic stem cells into the pancreas.

33. The method of claim 31 comprising transplantation of a cell population comprising insulin producing cells derived from human embryonic stem cells to an ectopic site.
